(19) United States
(12) Patent Application Publication
(10) Pub. No.: US 2026/0102588 A1
(43) Pub. Date: Apr. 16, 2026

(54) CONTINUUM MANIPULATOR AND SYSTEM COMPRISING SUCH

(71) Applicant: UNIVERSITEIT TWENTE, Enschede (NL)

(72) Inventors: Michiel RICHTER, Enschede (NL); Sarthak MISRA, Enschede (NL); Venkatasubramanian KALPATHY VENKITESWARAN, Enschede (NL)

(21) Appl. No.: 19/120,721

(22) PCT Filed: Oct. 13, 2023
(86) PCT No.: PCT/NL2023/050537

§ 371 Date: Apr. 11, 2025

(30) Foreign Application Priority Data
Oct. 13, 2022 (NL) 2033298

(51) Int. Cl. A61M 25/01; A61B 17/00; A61B 34/00; A61B 34/30

(52) U.S. Cl. CPC A61M 25/0158; A61B 34/30; A61B 34/73; A61B 2017/00318; A61B 2017/00876; A61B 2034/301; A61B 2034/731

(57) ABSTRACT

A continuum manipulator includes an elongated body defining a virtual axis extending in a longitudinal direction. The elongated body includes at least two magnets, distributed along the virtual axis and arranged such that the magnetic fields of these two magnets are oriented along the virtual axis, at least one flexible segment, each arranged in between two neighboring magnets and having a length defining a distance between two neighboring magnets. A resilience of the at least one segment and the mutual attractive force active over the distance between the two neighboring magnets are configured such that the elongated body is predisposed to flex under influence of an externally applied first magnetic field comprising a constant component transverse to the magnetic fields of the at least two magnets, and to stretch out in the longitudinal direction in the absence of the first externally applied magnetic field.

However, materials displaying an increased magnetic moment may also have an increased stiffness, and therefore may still require an increase in magnetic force to be bent in the first place. The increased stiffness may indicate that the increase in magnetic moment may not be as advantageous as expected, or, even when taking into account the larger magnetic moment, the flexibility of a continuum manipulator made of such material may be reduced. Vice versa, a gained flexibility inherent to a slender design may compensate for the reduction in torque that may be induced. However, the reduction in axial magnetic force is not compensated by magnetic continuum manipulator design. Accordingly, there is a trade-off in design for flexibility required to maneuver narrow pathways and amount of magnetic moment for ease of manipulation to move along those pathways.

### SUMMARY OF INVENTION

It is an object of the invention to provide a continuum manipulator having a magnetic moment sufficient for magnetic forces to deform and/or displace the continuum manipulator, while being sufficiently flexibility to navigate tortuous environments.

The abovementioned object is at least partially achieved in a continuum manipulator according to claim 1. The disclosure provides a continuum manipulator comprising an elongated body, an axis of which extends in a longitudinal direction of the elongated body. The elongated body comprises at least two magnets and at least one flexible segment. The at least two magnets are distributed along the axis defined by the elongated body and are arranged such that two neighboring magnets face each other with opposite poles. Of the at least one flexible segment, each flexible segment is arranged in between two neighboring magnets of the at least two magnets.

An example of a material able to address the above-mentioned trade-off may be a magnetic polymer composite (MPC), which is a polymer in which magnetic particles are suspended. MPC's with low density of magnetic particles are flexible but have low magnetic moment. Whereas MPC's with large density of magnetic particles have high magnetic moment, but are also more stiff. The proportional terms used to describe either of these MPC's (e.g. low/high, flexible/stiff) are with respect to the other.

Further objects, aspects, effects and details of particular embodiments of the invention are described in the following detailed description of a number of exemplary embodiments, with reference to the drawings.

### BRIEF DESCRIPTION OF DRAWINGS

By way of example only, the embodiments of the present disclosure will be described with reference to the accompanying drawings, wherein:

FIG. 1 illustrates schematically an example of a continuum manipulator in accordance with the invention;

FIG. 2 is a perspective view of the continuum manipulator of FIG. 1;

FIG. 3 illustrates schematically components of the continuum manipulator of FIG. 1;

FIG. 4 illustrates schematically an example of a continuum manipulator in a magnetic field having a transverse component;

FIG. 5 illustrates schematically an example of a continuum manipulator in magnetic field having a gradient component;

FIGS. 6A and 6B illustrate a perspective view and a side view of another example of a flexible segment in accordance with the invention;

FIG. 7 illustrates schematically another example of a continuum manipulator in accordance with the invention;

FIG. 8 illustrates schematically another example of a continuum manipulator in accordance with the invention;

FIG. 9 illustrates schematically a further example of a continuum manipulator in accordance with the invention;

FIG. 10 illustrates schematically yet another example of a continuum manipulator in accordance with the invention; and

FIG. 11A illustrates schematically an alignment torque and FIG. 11B illustrates schematically a misalignment torque.

### DETAILED DESCRIPTION

Referring to FIGS. 1, 4 and 5 a continuum manipulator 1 is shown having an elongated body 6 which defines a virtual axis 7 extending in a longitudinal direction. The elongated body 6 includes at least two magnets 2, in this example nine magnets, which are distributed along the virtual axis 7. The magnets are arranged such that the magnetic fields 8 of the at least two magnet s are oriented along the virtual axis 7. The elongated body 6 further includes at least one flexible segment 3, in this example eight, each flexible segment 3 being arranged in between two neighboring magnets 2 of the at least two magnets 2. Each segment 3 having a length L which defines a distance between two neighboring magnets 2.

Each segment 3 further has a particular resilience, or reciprocally a corresponding stiffness. The resilience of the at least one segment 3 and the mutual attractive force, active over the distance, between the at least two magnets 2 are configured such that the elongated body 1 is predisposed to flex under influence of an externally applied first magnetic field 9 having a constant component transverse to the magnetic fields 8 of the at least two magnets 2.

The resilience of the at least one segment 3 and the mutual attractive force, active over the distance between two neighboring magnets, between the at least two magnets 2 are further configured such that the elongated body 1 is predisposed to stretch out in the longitudinal direction in the absence of the first externally applied magnetic field 9.

Two neighboring magnets may be arranged such that they face each other with opposite poles. Or they may be arranged to face each other with same poles. The orientation of the magnets, their poles and corresponding magnetic field lines, will determine whether their interaction enhances flexibility of the continuum manipulator, as when facing with opposite poles, or increase the stiffness, as when facing with the same poles, of the continuum manipulator. Accordingly, the magnets may all be oriented in the same way or they may be oriented facing alternatingly with same and opposite poles. Alternatively, some of the magnets may be oriented facing with opposite poles, while some at e.g. a regular interval are oriented facing with same poles. For example, two or three consecutive magnets may be oriented to face with opposite poles, while e.g. every third or respectively fourth magnet may be oriented facing with the same poles. Various further configurations may be contemplated, such as facing intermittently i.e. with irregular intervals with same or opposite poles.

Similar to orientation, also the magnetic field strength of the at least two magnets may be chosen such as to provide an optimum between resilience and stiffness depending on a type of application. To that end, in some embodiments, the magnets may be permanent magnets of different size and/or material, such as e.g. neodymium-iron-boron (NdFeB) ring magnets. In addition, some of the at least two magnets may be electromagnets. The magnets are preferably embodied as dipole magnets, as this may allow ease of controlling magnetic fields and predicting maneuverability. As electromagnets require a current, this may be provided by electrical wiring, which may run through a backbone. In embodiments with electromagnets, the magnetic field of these electromagnets may be controlled via the current that is provided.

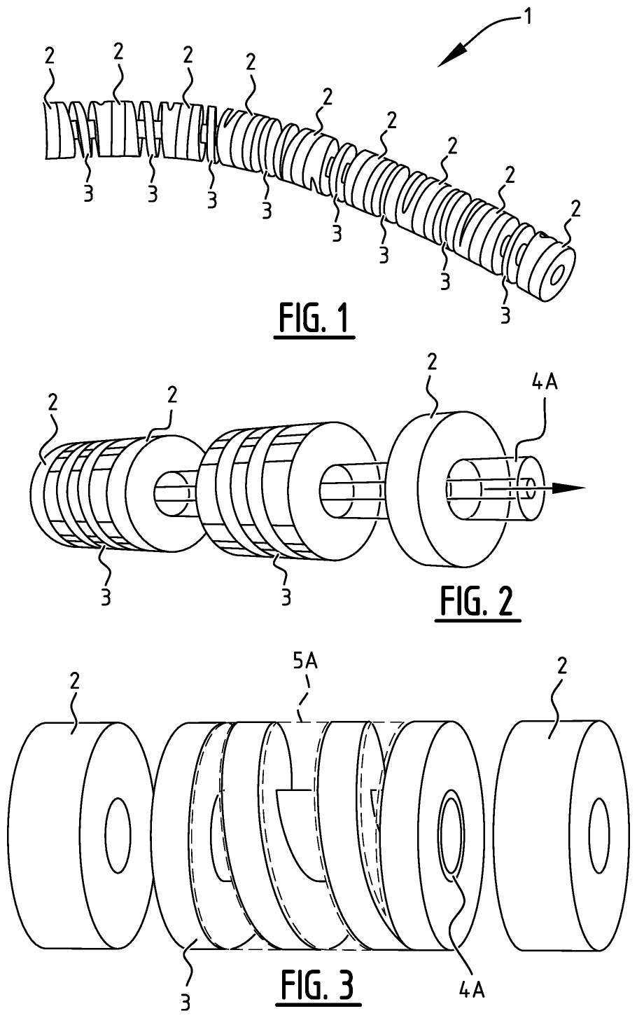

Referring to FIG. 2, the continuum manipulator has an elongated backbone element 4, such as a tube or a wire. In the example of FIG. 2, the backbone element is a tube 4A with an inner lumen 4B. The diameter of the inner lumen may be configured such that it allows passage of a e.g. a guide wire or other surgical instrument. It may also be configured for delivery of a particular substance, such as a medical medicament. In another example, the backbone element may include multiple lumen. Accordingly, the length of the backbone element 4 is preferably longer than the elongated body 1, such that it may be used to deliver such instruments or substances over a larger distance. Thereto one end-part of the backbone element 4 runs at least partially through, and preferably completely through the elongated body in lengthwise direction. With the use of multiple lumen, electrical wiring may be laid out via such lumen.

Referring to FIG. 3, two neighboring magnets 2 and one in between segment 3 are shown as separate components. The segment 3 has a helical shape which provides the segment flexibility. In addition, in this example, the segment has an inner tube-like core to provide for structural support.

The magnets may be dipole magnets, i.e. having one north and one south pole as known in magnetism. The magnets may have a cylindrical shape, a disc-like shape, or a ring-like shape. A central axis of such shapes may substantially align with, and more preferably coincide with the axis defined by the elongated body. Furthermore, the magnets may have substantially flat and/or planar shape, with a thickness of said shape substantially aligning the axis defined by the elongated body.

Whatever the shape of the magnets and of the segment, these are to be arranged such that magnetic field lines of the magnets are aligned with the virtual axis of the elongated body. Providing a magnetic field substantially parallel aligned and extending along the axis of the elongated body. As the elongated body is intended to flex under influence of an external magnetic field that is applied, while also showing an inclination to stretch or straighten in longitudinal direction in absence of the magnetic field, this in order to be able to steer the continuum manipulator along a tortuous path, such as e.g. in a venous system.

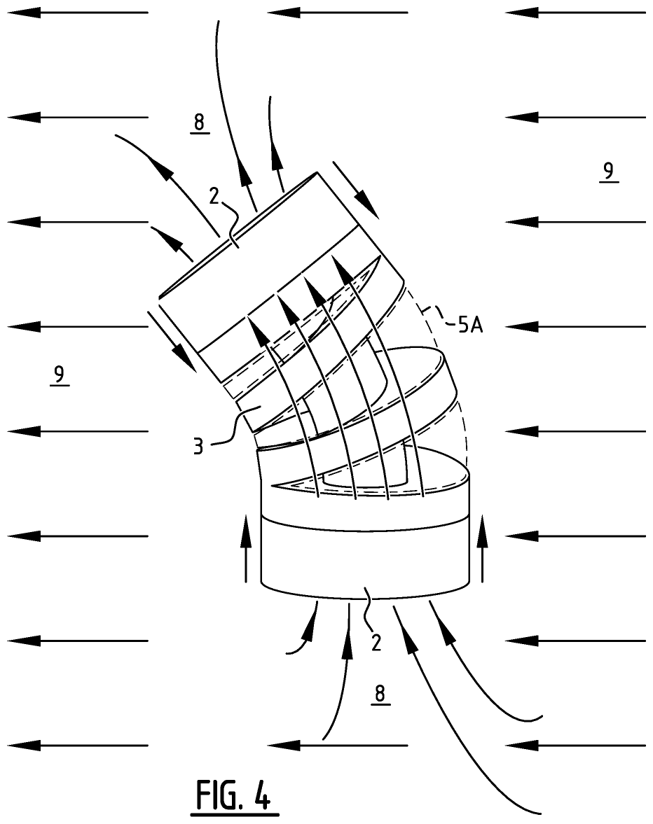

Referring to FIG. 4, the continuum manipulator, or a part thereof, made of components as in FIG. 3, is shown in an example of the first magnetic field 9, represented as a vector field, having a constant component that is transverse to the magnetic field 8 between the magnets 2. When positioned in a tortuous path, or another channel limiting the freedom of movement of the continuum manipulator, the elongated body will experience a force due to the influence of this first magnetic field that will cause it to flex and thus may be manipulated i.e. steered by applying the first magnetic field to bend and turn around a corner.

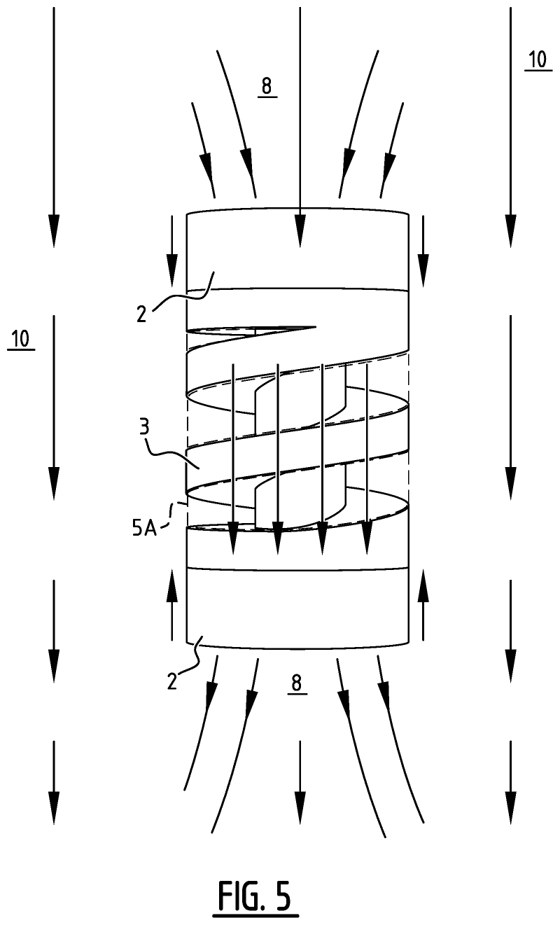

Referring to FIG. 5, the continuum manipulator, or a part thereof, made of components as in FIG. 3, is shown in an example of a second magnetic field 10 having a gradient component that is parallel to the magnetic field 8 between the magnets 2. The gradient of the second magnetic field 10 is represented by a vector field of vectors shortening in length. When such second magnetic field is applied, the elongated body will experience a force that will cause it to be pulled, or pushed as may be the case, and thus may be manipulated i.e. steered by applying the second magnetic field to move in a straight direction.

As will be understood, such first 9 and second magnetic fields 10 having constant transverse and gradient parallel components may be applied in combination in order to steer the whole continuum manipulator through a maze or along a tortuous path towards a desired location or position. Systems for providing such combined magnetic fields, referred to as magnetic actuators, are known per se. These may for example include an articulated robotic arm carrying one or more electromagnetic elements that can be positioned in various orientations thereby allowing to move and steer the continuum manipulator.

The resilience of the segment 3 and the mutual attractive force between the at least two magnets 2, which attractive force acts over the distance determined by the length of the segment, are configured such that the elongated body 1 is predisposed to flex under influence of the externally applied first magnetic field 9 having a constant component transverse to the magnetic fields 8 of the at least two magnets 2. In addition, the resilience of the at least one segment 3 and the mutual attractive force between the at least two magnets 2 are further configured such that the elongated body 1 is predisposed to stretch out in the longitudinal direction in the absence of the first externally applied magnetic field 9.

When the first, transverse, magnetic field is applied and the elongated body starts flexing i.e. bending to one side, a dipole action between the magnets enhances the flexing, as the curvature brings the magnets at one side of elongated body 1 closer together. As a result, the mutual attraction between the magnets increases. While when removing, or in absence of, the first magnetic field the segment due to its stiffness, or reciprocally its resilience, will stretch and straighten out, or recoil so to say.

Accordingly, the flexible segment is configured to limit the amount of curvature of a part of the axis between the at least two magnets when the elongated body flexes under influence of the externally applied magnetic field which includes a constant component. While the flexible segment is further configured to maintain a minimum distance between the at least two magnetic fields. This to prevent that the magnets will connect and attach to one another, making it perhaps impossible to separate them again. As may be understood, in a preferred embodiment, the flexible segment is fixated to at least one of the at least two neighboring magnets in between which flexible segment is arranged.

In order to further enhance the stiffness of the segment, and at the same time enhance the susceptibility of the elongated body to an external magnetic field, the segment may be magnetized itself. In one manner, the segment may include a plurality of magnetic particles, such that the segment obtains or provides a magnetic moment. The amount of and distribution of particles may be configured such that stiffness, or vice versa resilience, is optimized in relation to the mutual attractive force between the magnets. In some embodiments, the flexible segment may be made of a magnetic polymer or magnetic polymer composite. A magnetic polymer composite may for example be provided by a suspension of polydimethylsiloxane PDMS and praseodymium-iron-boron PrFeB microparticles.

In a preferred embodiment, the at least two magnets 2 are provided in the same way as the magnetized elongated body. That is, the at least two magnets 2 may include a plurality of magnetic particles or made of a magnetic polymer or magnetic polymer composite, such as the ones mentioned above in relation to the magnetized elongated body.

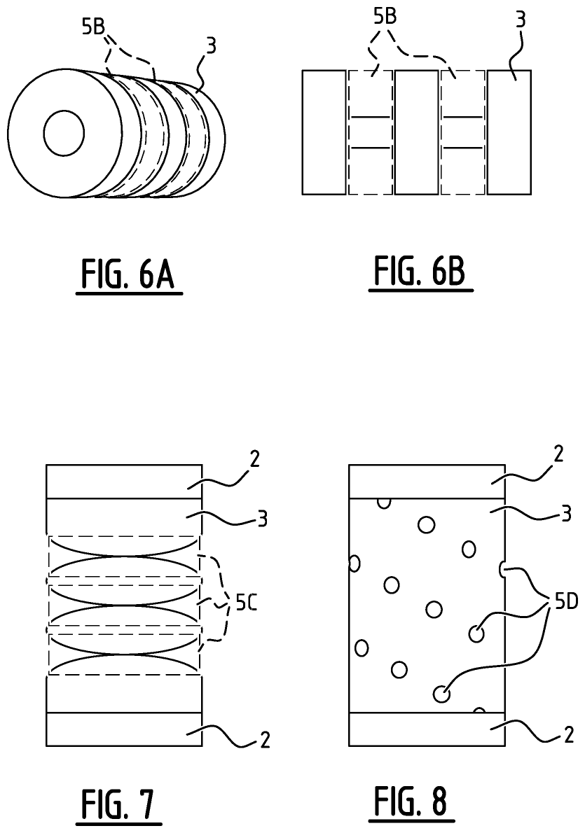

The flexible segment of FIG. 3 is just one example of segment 3 suitably configured. More in general, the segment may be defined by a first surface, substantially parallel to the axis defined by the elongated body, and wherein at least one indentation, such as a recess, nick, or notch, is provided in said first surface. The indentation 5A may have a helical shape, as in FIG. 3, or an annular shape 5B, as in FIGS. 6A and 6B. For each indentation shape the axis of said shape substantially coincides with the axis defined by the elongated body.

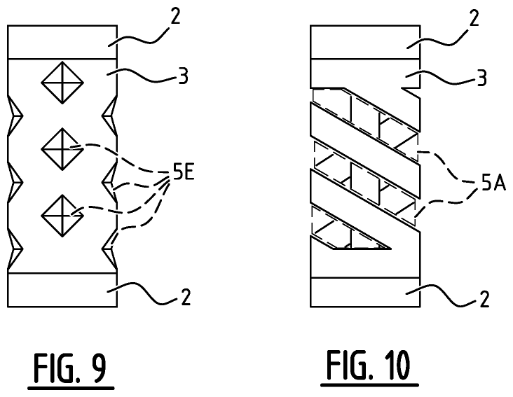

Referring to FIGS. 8 and 9, the indentation may include a plurality of indentations 5D, 5E, distributed over a circumference of the flexible segment 3 such that, when seen from the lengthwise direction of the elongated body 1, at least part of the indentation is present at each circumferential position on the surface with respect to the axis defined by the elongated body.

In some embodiments, the flexible segment may be substantially shaped like a cylinder, or a pillar having three or more sides. Of which shapes an axis substantially aligns with, and preferably coincides with the axis defined by the elongated body.

The segment in turn may include a plurality of flexible segments, wherein a cross-section of each segment in a plane spanned by a vector in the lengthwise direction of the elongated body and a vector perpendicular thereto, has either a rectangular, a circular, an ellipsoidal, or a lenticular shape. Examples thereof are shown in FIGS. 7-10. In other embodiments, the flexible segment itself may be defined as having a substantially helical shape, an axis of which substantially coincides with the axis defined by the elongated body.

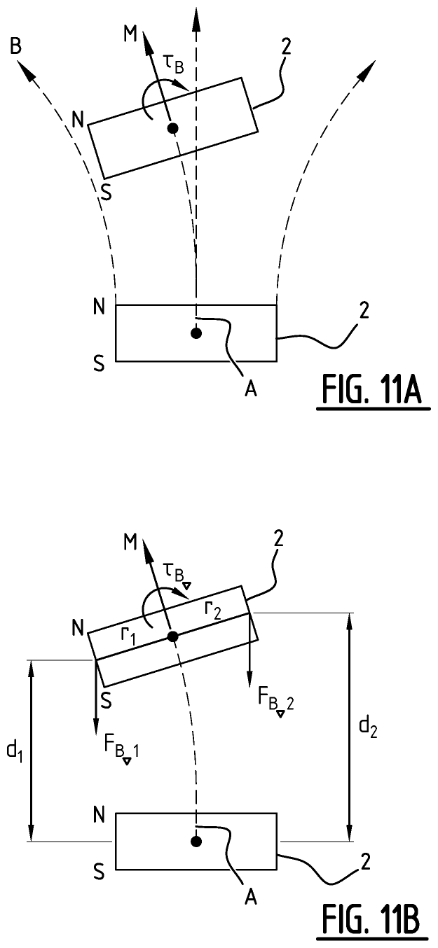

FIG. 11A illustrates the at least two magnets 2 of an embodiment of a continuum manipulator and, in each of them, a dot indicates their magnetic center. Polarities are indicated for each of the at least two magnets 2: N for north and S for south. The virtual axis A extends from one of the at least two magnets 2 to the other. For a first magnet (the lower magnet 2 in FIG. 11A) magnetic field lines B are shown. For a second magnet (the upper magnet 2 in FIG. 11A) the magnetic moment M is shown.

While not shown in FIG. 11A, at least one flexible segment 3 is arranged between the first magnet 2 and second magnet 2.

In FIG. 11A, the two magnets 2 are not parallel to each other. In such a configuration, due to magnetic field B and magnetic moment M, the second magnet experiences an alignment torque τ_{B}. This alignment torque urges the two magnets to align and, since the at least one flexible segment 3 is arranged between the two magnets, urges the elongated body to stretch out in the longitudinal direction.

While not shown in FIG. 11A, the first magnet also has a magnetic moment and the second magnet also has a magnetic field so in the configuration shown in FIG. 11A the first magnet also experiences an alignment torque τ_{B}.

FIG. 11B illustrates the at least two magnets 2 also discussed in relation to FIG. 11A. The first magnet is the lower magnet 2 in FIG. 11B. The second magnet is the upper magnet 2 in FIG. 11B. The virtual axis A extends from one of the at least two magnets to the other. While not shown in FIG. 11B, at least one flexible segment 3 is arranged between the first magnet 2 and second magnet 2.

Between two magnets, a mutual attractive force exists. In FIG. 11B, the attractive force of the first magnet on the second magnet is shown. The force F_{B}_{_{∇}}_{¹} indicates the part of the attractive force of the first magnet 2 acting on the left side of second magnet 2, over moment arm r₁. The force F_{b}_{_{∇}}_{²} indicates the part of the attractive force of the first magnet 2 acting on the right side of the second magnet 2, over moment arm r₂. While not shown, the second magnet also has an attractive force that acts upon the first magnet in a similar way.

The mutual attractive force is a function of the distance between two magnets. In FIG. 11B, di indicates the distance between the left side of the first magnet 2 and the left side of the second magnet 2 and d₂ indicates the distance between the right side of the first magnet and the right side of the second magnet 2.

When the elongated body is stretched out and the at least two magnets 2 are parallel to each other, distances d₁ and d₂ are approximately equal and forces F_{B}_{_{∇}}_{¹} and F_{B}_{_{∇}}_{²} are approximately equal. However, corresponding to the configuration shown in FIG. 11A, in FIG. 11B the two magnets 2 are not parallel to each other. Instead, d₁ is smaller than d₂ and force F_{B}_{_{∇}}_{¹} is larger than F_{B}_{_{∇}}_{²}. The difference between the attractive forces F_{B}_{_{∇}}_{¹} and F_{B}_{_{∇}}_{²} results in a misalignment torque τ_{B}_{_{∇}}.

This misalignment torque τ_{B}_{_{∇}} urges the two magnets to further misalign, and, since the at least one flexible segment 3 is arranged between the two magnets, urges the elongated body to flex. This flexing of the elongated body in turn increases the mutual attractive force and the further misalignment increases the relative difference between d₁ and d₂. These factors may contribute to an increase in F_{B}_{_{∇}}_{¹} and F_{B}_{_{∇}}_{²}, and/or a stronger misalignment torque τ_{B}_{_{∇}}.

In the frame of reference used in FIG. 11A and FIG. 11B the clockwise direction is the positive direction. This direction corresponds to the direction in which the arrow indicating the alignment torque and the arrow indicating the misalignment torque points. In the embodiments shown in FIGS. 11A and 11B, the alignment torque τ_{B} will therefore have a positive value and the misalignment torque τ_{B}_{_{∇}} will have a negative value.

While not shown in FIG. 11B, the second magnet also has an attractive force that acts on the first magnet so in the configuration shown in FIG. 11B the first magnet also experiences a misalignment torque τ_{B}_{_{∇}}.

In a continuum manipulator according to the invention, the mutual attractive force active over the distance between the two neighboring magnets is configured such that, when the elongated body is stretched, the alignment torque (explained in FIG. 11A) is larger than the misalignment torque (explained in FIG. 11B). Additionally, the resilience of the at least one segment resist misalignment.

This predisposes the elongated body to stretch out in the longitudinal direction in the absence of the first externally applied magnetic field.

Without wishing to be bound by theory, the applicant finds that the alignment torque is a function of the distance between the at least two magnets 2 following 1/d³ and that the misalignment torque is a function of the distance between the at least two magnets 2 following 1/d⁴. That is, when the distance between the at least two magnets 2 decreases, the misalignment torque increases faster than the alignment torque. And, vice-versa, when the distance between the at least two magnets 2 increases, the alignment torque increases faster than the misalignment torque.

When an external magnetic field with a constant component transverse to the magnetic fields of the at least two magnets is applied, the at least two magnets both experience a torque τ_{B} from magnetic field urging them to align their magnetic fields with it. The at least two magnets misalign, the elongated body flexes, and the at least two magnets move closer to each other.

In a continuum manipulator according to the invention, the mutual attractive force active over the distance between the two neighboring magnets is configured such that, when the elongated body flexes, the alignment torque (explained in FIG. 11A) is smaller than the misalignment torque (explained in FIG. 11B).

This predisposes the elongated body to flex under the influence of said externally applied magnetic field.

Although the present invention has been described above with reference to specific embodiments, it is not intended to be limited to the specific form set forth herein. Rather, the invention is limited only by the accompanying claims and, other embodiments than the specific above are equally possible within the scope of these appended claims.

Furthermore, although exemplary embodiments have been described above in some exemplary combination of components and/or functions, it should be appreciated that, alternative embodiments may be provided by different combinations of members and/or functions without departing from the scope of the present disclosure. In addition, it is specifically contemplated that a particular feature described, either individually or as part of an embodiment, can be combined with other individually described features, or parts of other embodiments.

1. A continuum manipulator, comprising:
an elongated body, defining a virtual axis extending in a longitudinal direction, the elongated body comprising:
at least two magnets, distributed along the virtual axis and disposed such that magnetic fields of the at least two magnets are oriented along the virtual axis, and
at least one flexible segment, each flexible segment of the at least one flexible segment being disposed in between two neighboring magnets of the at least two magnets, each flexible segment of the at least one flexible segment having a length defining a distance between two respective ones of the neighboring magnets,
wherein a resilience of the at least one flexible segment and a mutual attractive force active over the distance between the two respective neighboring magnets are configured such that the elongated body is predisposed to:
flex under an influence of an externally-applied first magnetic field comprising a constant component transverse to the magnetic fields of the at least two magnets, and
stretch out in the longitudinal direction in an absence of the first externally-applied magnetic field.

2. The continuum manipulator according to claim 1, wherein the resilience of the at least one flexible segment and the mutual attractive force between the two respective neighboring magnets active over the distance are further configured such that the elongated body experiences a pulling force when an externally-applied second magnetic field comprises a gradient component parallel to the magnetic fields of the at least two magnets.

3. The continuum manipulator according to claim 1, wherein two neighboring magnets of the at least two magnets face each other with same magnetic poles or opposite magnetic poles.

4. The continuum manipulator according to claim 1, wherein the at least one flexible segment is one or more of magnetized and includes a plurality of magnetic particles, such that the segment provides a magnetic moment.

5. The continuum manipulator according to claim 1, further comprising an elongated backbone piece having a length that is longer than the elongated body, and
wherein one end-part of the elongated backbone piece runs at least partially through the elongated body in the lengthwise direction.

6. The continuum manipulator according to claim 1, wherein at least one magnet of the at least two magnets is a permanent magnet or an electromagnet.

7. The continuum manipulator according to claim 1, wherein the at least two magnets are disposed such that magnetic field lines therethrough are aligned with the virtual axis of the elongated body.

8. The continuum manipulator according to claim 1, wherein at least one magnet of the at least two magnets has one of: a cylindrical shape, a disc-like shape, and a ring-like shape, and
wherein an axis of the one of said cylindrical shape, the disc-like shape, and the ring-like shape substantially aligns with the virtual axis defined by the elongated body.

9. The continuum manipulator according to claim 1, wherein at least one magnet of the at least two magnets has a substantially flat and/or planar shape, and
wherein a thickness of said substantially flat and/or planar shape substantially aligns with the virtual axis defined by the elongated body.

10. The continuum manipulator according to claim 1, wherein the at least one flexible segment is configured to one or more of: (i) limit an amount of curvature of a part of the virtual axis between the at least two magnets when the elongated body flexes under the influence of the externally-applied magnetic field comprising a constant component, and (ii) maintain a minimum distance between the at least two magnetic fields.

11. The continuum manipulator according to claim 1, wherein the at least one flexible segment comprises a first surface, substantially parallel to the virtual axis defined by the elongated body, and
wherein at least one indentation is provided in said first surface.

12. The continuum manipulator according to claim 11, wherein the at least one indentation has one of a helical shape and an annular shape, and wherein an axis of said one of the helical shape and the annular shape substantially coincides with the virtual axis defined by the elongated body.

13. The continuum manipulator according to claim 11, wherein the at least one indentation comprises a plurality of indentations, distributed over a circumference of the at least one flexible segment such that, when seen from the longitudinal direction of the elongated body, at least part of at least one of the indentations is present at each circumferential position with respect to the virtual axis defined by the elongated body.

14. The continuum manipulator according to claim 1, wherein the at least one flexible segment is substantially shaped as one of a cylinder and a pillar having three or more sides, and
wherein an axis of said one of the cylinder and the pillar substantially aligns with the virtual axis defined by the elongated body.

15. The continuum manipulator according to claim 1, wherein the at least one flexible segment comprises a plurality of flexible segments,
wherein a cross-section of each of the flexible segments in a plane spanned by a vector in the lengthwise direction of the elongated body and a vector perpendicular thereto, has one of a rectangular shape, a circular shape, an ellipsoidal shape, and a lenticular shape.

16. The continuum manipulator according to claim 1, wherein the at least one flexible segment has a substantially helical shape, and
wherein an axis of said substantially helical shape substantially coincides with the virtual axis defined by the elongated body.

17. The continuum manipulator according to claim 1, wherein the at least one flexible segment is made of a magnetic polymer.

18. The continuum manipulator according claim 1, wherein a first flexible segment from the at least one flexible segment is fixated to at least one magnet of the at least two neighboring magnets in between which said first flexible segment is disposed.

19. A system comprising:
the continuum manipulator according to claim 1; and
one or more magnetic actuators configured and/or disposed to generate an electromagnetic field which, when arranged therein, bends and/or displaces the continuum manipulator.

20. The continuum manipulator according to claim 7, wherein the magnetic field lines at least partially coincide with the virtual axis of the elongated body.
